(19) United States
(12) Patent Application Publication
(10) Pub. No.: US 2023/0414474 A1
(43) Pub. Date: Dec. 28, 2023

(54) PROCESS FOR MAKING A COMPOSITION COMPRISING ALKYL POLYGLYCOSIDE AND PERFUME

(71) Applicant: The Procter & Gamble Company, Cincinnati, OH (US)

(72) Inventors: Jean-Francois Bodet, Waterloo (BE); Geert Andre Deleersnyder, Wielsbeke (BE); Cindy Jean, Houdeng-Aimeries (BE)

(21) Appl. No.: 18/340,949

(22) Filed: Jun. 26, 2023

(30) Foreign Application Priority Data
Jun. 27, 2022 (EP) 22181218.3

(51) Int. Cl. A61K 8/60; A61Q 13/00

(52) U.S. Cl. CPC A61K 8/604; A61Q 13/00; A61K 2800/596; A61K 2800/805

(57) ABSTRACT

A process for making a composition including providing a concentrated aqueous solution comprising from about 30 to about 80 wt. % alkyl polyglycoside and adding a neutralizing agent to the concentrated aqueous solution to form a mixture. The process further includes adding perfume to the mixture to form the composition. The composition includes alkyl polyglycoside surfactant and at least about 0.1% perfume by weight of the composition. The perfume comprises at least about 10 wt. % perfume raw material. The perfume raw material comprises an ester functionality, an aldehyde functionality, a ketone functionality, or combinations thereof, and the composition has a pH equal to or greater than about 7, as measured at about 25° C.

### TECHNICAL FIELD

The present disclosure relates to a process for making a composition, the composition comprising an alkyl polyglycoside surfactant and a perfume. It also relates to a composition obtainable by the process of the present disclosure, a method of using the composition and the use of a neutralizing agent to reduce perfume degradation in a composition comprising an alkyl polyglycoside surfactant and a perfume.

### BACKGROUND

Users constantly look for simplification of the cleaning task. Some cleaning processes involve using a cleaning product and rinsing it. Cleaning habits are changing. In the past, time was dedicated to do deep cleaning, recently, people seem to prefer to clean little and often and try to maintain surfaces clean, in order to avoid the build-up of dirt, and to facilitate the subsequent cleaning tasks. Users nowadays also expect pleasant smell and shine of the surface cleaned. A dichotomy exists with aqueous cleaning compositions, the more perfume in the composition, the more emulsifier is required and this can negatively impact shine. It can also be difficult to stabilize a relatively high level of perfume in an aqueous composition, especially in alkaline compositions.

It is desirable to have a perfumed alkaline composition comprising an alkyl polyglycoside surfactant however, perfumes can easily degrade on alkaline compositions comprising an alkyl polyglycoside surfactant.

Alkyl glycosides are generally produced by reacting an alcohol with a reducing saccharide in the presence of an acid catalyst. The resulting solution is usually neutralized with an alkali, usually NaOH. Alkyl glycosides are soluble and stable in highly alkaline formulations. Usually, alkyl glycosides are supplied in the form of an aqueous alkaline solution ranging from 50 to 70% active. Alkyl glycosides solutions can negatively interact with perfumes components.

A need remains for an alkaline stable composition comprising alkyl polyglycoside surfactant and relatively high level of perfume. There is also a need for a composition which provides improved scent during use and improved shine. There is also a need for a simplified method of cleaning a surface.

### SUMMARY

According to the first aspect of the present disclosure, there is provided a process for making a composition, the composition comprising:
a) an alkyl polyglycoside surfactant, preferably an alkyl polyglucoside; and
b) at least 0.1% by weight of the composition of a perfume wherein the perfume comprises at least 10% by weight of the perfume of perfume raw materials, the perfume raw materials comprising an ester functionality, an aldehyde functionality, a ketone functionality or a mixture thereof;
   the composition has a pH equal or greater than about 7, preferably equal or greater than about 10 as measured at 20° C., the process comprises the steps of:
i) providing a concentrated aqueous solution comprising from 30 to 80% by weight of the solution of the alkyl polyglycoside;
ii) adding a neutralizing agent to the solution resulting from step i);
iii) adding the perfume to the mixture resulting from step ii).

It has surprisingly been found that the process of the present disclosure gives rise to a composition in which the perfume is stable. The addition of the neutralizing agent contributes to the chemical stability of the perfume.

According to the second aspect of the present disclosure, there is provided a composition obtainable or obtained according to the process of the present disclosure.

According to the third aspect of the present disclosure, there is provided a method of treating a surface, preferably, a hard surface. The method comprises the steps of treating the surface with the composition of the present disclosure and wiping the surface. The method of the present disclosure does not require the step of rinsing. The method provides good cleaning and improved shine and more intensive perfume than methods using traditional hard surface compositions.

According to the fourth aspect of the present disclosure, there is provided the use of a neutralizing agent in the process of the present disclosure to reduce perfume degradation.

The elements of the composition made according to the process of the present disclosure described in relation to the first aspect of the present disclosure apply mutatis mutandis to the other aspects of the present disclosure.

### DETAILED DESCRIPTION

Compositions made according to the process of the present disclosure present good perfume stability, provide good cleaning, improved shine and very pleasant olfactory experience.

The composition of the present disclosure is preferably clear and the perfume has good chemical stability. The composition of the present disclosure preferably is isotropic and stable.

As defined herein, “essentially free of” a component means that no amount of that component is deliberately incorporated into the respective premix, or composition. Preferably, “essentially free of” a component means that no amount of that component is present in the respective premix, or composition.

As used herein, “isotropic” means a clear mixture, having little or no visible haziness, phase separation and/or dispersed particles, and having a uniform transparent appearance.

As defined herein, “physically stable” means that no visible phase separation is observed for a composition kept at 25° C. for a period of at least two weeks, or at least four weeks, or greater than a month.

All percentages, ratios and proportions used herein are by weight percent of the composition, unless otherwise specified. All average values are calculated “by weight” of the composition, unless otherwise expressly indicated.

All measurements are performed at 25° C. unless otherwise specified.

Unless otherwise noted, all component or composition levels are in reference to the active portion of that component or composition, and are exclusive of impurities, for example, residual solvents or by-products, which may be present in commercially available sources of such components or compositions.

### Process of the Present Disclosure

The process of the present disclosure comprises the steps of:
i) providing a concentrated aqueous solution, the concentrated aqueous solution comprises from 30 to 80%, preferably from 40% to 70% by weight of the solution of an alkyl polyglycoside surfactant, preferably an alkyl polyglucoside, more preferably a C8-10 alkyl polyglucoside;
ii) adding a neutralizing agent, preferably citric acid, to the solution of step i);
iii) adding the perfume to the mixture resulting from step ii).
   The concentrated aqueous solution comprising the alkyl polyglycoside surfactant usually has a starting pH of about 12 as measured at 25° C. The neutralizing agent, preferably citric acid, is added and mixed to allow neutralization to occur in order to bring the pH to 9 or less as measured at 25° C. Preferably the pH of the starting concentrated aqueous solution is lowered, after adding the neutralizing agent, by about 1 or 2 or 3 or 4 or 5 pH units, more preferably the pH of the concentrated aqueous solution is from about 7 to about 8. Usually, the amount of neutralizing agent required is from 0.2 to 5%, more preferably from 0.4 to 2% by weight of the concentrated aqueous solution.

Preferably, the neutralizing agent also acts as a chelating agent for transition metals. Transitions metals, such as iron, can be present in the initial concentrated aqueous solution as a byproduct of the synthesis of the alkyl polyglycoside surfactant. Chelation of transition metals may help the stability of the perfume.

The neutralizing agent may comprise an organic acid, an inorganic acid, or a mixture thereof. The neutralizing agent may be substantially free of trace transition metal impurities. A preferred organic acid of use herein has a pKa of less than 6. A suitable organic acid is selected from the group consisting of: citric acid, lactic acid, glycolic acid, succinic acid, glutaric acid and adipic acid and mixtures thereof. The preferred neutralizing agent for use herein is citric acid. A suitable inorganic acid can be selected from the group consisting of: hydrochloric acid, sulphuric acid, phosphoric acid and mixtures thereof. Citric acid is the preferred acid for use as neutralizing agent herein.

Suitable inorganic acids include phosphoric acid, sulfuric acid, urea-sulfuric acid, hydrochloric acid, sulfamic acid, methyl sulfuric acid, hypochlorous acid, sodium bisulfate, and the like. Suitable organic acids include polymeric acids comprising at least 3 carboxylic acid groups, C₁-C₁₁ organic acids comprising at least one carboxylic acid group, and organic acids that do not comprise carboxylic acid functional groups (such as imidazole derivatives or phenolic or polyphenolic compounds). Non-limiting examples of polymeric acids include polymers of acrylic acid, methacrylic acid, maleic acid, or itaconic acid or copolymers of acrylic acid, methacrylic acid, maleic acid, itaconic acid, or mixtures thereof. Polymeric acids may be homopolymers or copolymers having a molecular weight of about 500 g/mol or greater. The polymeric acid may have a molecular weight ranging from about 500 g/mol to about 1,000,000 g/mol, or from about 500 g/mol to about 100,000 g/mol, or from about 1,000 g/mol to about 20,000 g/mol. Copolymers may be random copolymers or block copolymers. In addition to monomer units comprising carboxylic acid groups, the copolymers may also include one or more other monomers, such as styrene, acrylic ester, acrylamide, olefin sulfonate, and olefin acetate.

Non-limiting examples of C₁-C₁₁ organic acids include formic acid, acetic acid, benzoic acid, malonic acid, citric acid, maleic acid, fumaric acid, succinic acid, lactic acid, malic acid, tartaric acid, gluconic acid, glutaric acid, adipic acid, 2-ethyl-1-hexanoic acid, heptanoic acid, octanoic acid, nonanoic acid, undecylenic acid, butane tetracarboxylic acid, and the like. The organic acid may be derived from a renewable, plant-based feedstock and produced using natural processes, such as fermentation; examples include bio-based acetic acid, bio-based citric acid, bio-based lactic acid and bio-based succinic acid, and the like. The organic acid may have food-use pedigree or be Generally Regarded As Safe (GRAS) or a food additive by the US Food & Drug Administration.

The neutralizing agent can be selected from the group consisting of formic acid, acetic acid, benzoic acid, malonic acid, citric acid, maleic acid, fumaric acid, hypochlorous acid, succinic acid, gluconic acid, glutaric acid, lactic acid, 2-ethyl-1-hexanoic acid, octanoic acid, nonanoic acid, peracetic acid, peroctanoic acid, undecylenic acid, and mixtures thereof, or the neutralizing agent can be selected from the group consisting of benzoic acid, citric acid, lactic acid succinic acid, maleic acid, succinic acid, octanoic acid, and mixtures thereof. Specially preferred for use herein is citric acid.

The process of the present disclosure can comprise a step before step i) in which an alkaline solution is made, preferably the solution comprise an alkaline agent, more preferably monoethanol amine. The mixture resulting from step iii) is added to the alkaline solution, dyes and other cleaning adjuncts can then be added to the resulting mixture. The mixture is homogenized by mixing.

### Composition

The composition of the present disclosure is preferably a treatment composition, more preferably a cleaning composition, more preferably a hard surface cleaning composition and most preferable an aqueous hard surface cleaning composition.

By “aqueous hard surface cleaning composition”, it is meant herein a water based liquid composition for cleaning hard surfaces found in households, especially domestic households.

Surfaces to be cleaned include kitchens and bathrooms, e.g., floors, walls, tiles, windows, cupboards, sinks, showers, shower plastified curtains, wash basins, WCs, fixtures and fittings and the like made of different materials like ceramic, vinyl, no-wax vinyl, linoleum, melamine, glass, steel, kitchen work surfaces, any plastics, plastified wood, metal or any painted or varnished or sealed surface and the like. Household hard surfaces also include household appliances including, but not limited to refrigerators, freezers, washing machines, automatic dryers, ovens, microwave ovens, dishwashers and so on. Such hard surfaces may be found both in private households as well as in commercial, institutional and industrial environments. The composition of the present disclosure is especially suitable for the cleaning and treatment of kitchen surfaces.

The composition herein is an aqueous alkaline composition. By “aqueous composition” is herein meant a composition comprising more than 50%, preferably more than 60% by weight of the composition of water. The composition may comprise from 80% to 99.5%, preferably from 85% to 99% and more preferably from 94% to 98% by weight of the total composition of water.

The composition of the present disclosure has a pH which is greater or equal than 7, preferably greater or equal than 10, preferably greater than 10.5 as measured on the neat composition, at 25° C. It is believed that the greasy soil and particulate greasy soil cleaning performance is further improved at these preferred alkaline pH ranges, while surface safety is maintained. Accordingly, the compositions herein may further comprise a base to adjust pH as appropriate.

A suitable base to be used herein is an organic and/or inorganic base. Suitable bases of use herein are the caustic alkalis, such as sodium hydroxide, potassium hydroxide and/or lithium hydroxide, and/or the alkali metal oxides such, as sodium and/or potassium oxide or mixtures thereof. A preferred base is a caustic alkali, more preferably sodium hydroxide and/or potassium hydroxide.

Other suitable bases include ammonia, ammonium carbonate, potassium carbonate, sodium carbonate, sodium bicarbonate, and alkanolamines (such as monoethanolamine, triethanolamine, aminomethylpropanol, and mixtures thereof), nitrogenous buffers, and mixtures thereof. Suitable nitrogenous buffers include: ammonium or alkaline earth carbamates, guanidine derivatives, ammonium carbonate, ammonium bicarbonate, diammonium carbonate, ammonium hydroxide, ammonia (which forms ammonium hydroxide in situ when added to water) and mixtures thereof.

Typical levels of such bases, when present, are from 0.01% to 5.0% by weight of the total composition, preferably from 0.05% to 3.0% and more preferably from 0.1% to 2.0%.

All ratios are calculated as a weight/weight level, unless otherwise specified.

### Alkyl Polyglycoside Surfactant

The composition of the present disclosure comprises an alkyl polyglycoside (APG). Preferred APGs include alkyl polyglucosides, which are characterized by the saccharide moiety being glucose. Preferred alkyl polyglucosides have naturally derived glucoside groups.

The alkyl polyglycosides, which can be used in the present disclosure, are fatty ether derivatives of saccharides or polysaccharides which are formed when a carbohydrate is reacted under acidic condition with a fatty alcohol through condensation polymerization. The APGs commonly are derived from corn-based carbohydrates and fatty alcohols from natural oils in animals, coconuts and palm kernels. Natural gas, or petroleum-based alcohols may also be used, particularly in shorter chain lengths. Such methods of deriving APGs are known in the art, for example, U.S. Pat. No. 5,003,057.

The alkyl polyglycoside that can be used in the present disclosure contains a hydrophilic group derived from carbohydrates and is composed of one or more anhydroglucose. Each of the glucose units can have two ether oxygens and three hydroxyl groups and a terminal hydroxyl group, imparting water solubility to the glycoside. The presence of the alkyl carbons leads to the hydrophobic activity. When carbohydrate molecules react with fatty alcohol molecules, alkyl poly glycoside molecules are formed with single or multiple anhydroglucose units, which are termed monoglycosides and polyglycosides, respectively. The final alkyl poly glycoside product typically has a distribution of varying concentration of glucose units (or degree of polymerization).

The APG used in the present disclosure preferably comprises the saccharide or polysaccharide groups (i.e., mono-, di-, tri-, etc. saccharides) of hexose or pentose, and a fatty aliphatic group with 6 to 20 carbon atoms. Alkyl polyglycosides which can be used in the present disclosure are represented by the general formula of

(G)_{x-O-R}

where G is a moiety derived from a reducing saccharide containing 5 or 6 carbon atoms, e.g., pentose or hexose; R is fatty aliphatic group containing 6 to 20 carbon atoms; and x is the degree of polymerization (D.P.) of the polyglycoside, representing the number of monosaccharide repeating units in the polyglycoside. Generally, x is an integer on the basis of individual molecules, but because there are statistical variations in the manufacturing process of the APG, x may be a non-integer on an average basis when referred to APG used as an ingredient for composition of the present disclosure. In suitable APGs for use herein, x preferably has a value of less than about 5, and more preferably between about 0.5 and about 5. Even more preferably, x is less than about 2.5, and more preferably is within the range between about 1 and about 2.

Many commercially available alkyl polyglycosides may contain a blend of carbon lengths. Suitable alkyl polyglycosides include alkyl polyglycosides containing short chain carbons, such as chain lengths of less than C16. In one example, suitable alkyl polyglycosides include C8-C16 alkyl polyglycosides. Additional description of suitable alkyl polyglycosides are set forth, for example, in U.S. Pat. Nos. 8,287,659 and 8,299,009, and U.S. patent application Ser. Nos. 12/819,667, 12/884,638, 12/887,716, 13/597,380, 13/622,392, and 13/653,965.

Exemplary saccharides from which G is derived are glucose, fructose, mannose, galactose, talose, gulose, allose, altrose, idose, arabinose, xylose, lyxose and ribose. Because of the ready availability of glucose, glucose is preferred in the making of polyglycosides. The fatty aliphatic group, which is the substituent of the preferred polyglycoside, is preferably saturated, although unsaturated fatty group may be used.

In some embodiments, the APGs have an average degree of polymerization of saccharides from 1.4 to 1.7 and the chain lengths of the aliphatic groups are between C8-16. Alkyl polyglycosides suitable for this disclosure can be described as illustrated in the following way: “C8-16 G 1.6” denotes a polyglycoside with an alkyl chain of 8 to 16 carbon atoms and an average degree of polymerization of 1.6 anhydroglucose units in the alkyl polyglucoside molecule.

Commercially, alkyl polyglycosides can be provided as concentrated, aqueous solutions ranging from 50 to 70 wt. % active. Examples of suitable alkyl polyglucoside surfactants are the TRITON™ alkyl polyglucosides from Dow; Agnique PG, Disponil APG and Glucopon alkyl polyglucosides from BASF. Preferred alkyl polyglucoside surfactants are those where n is 8 to 12, more preferably 8 to 10. Examples of preferred polyglycosides include AG 6202, 2-ethylhexyl APG from Nouryon, Triton CG-50, C8-10 APG with low degree of glucose oligomerization from Dow, Triton GC-110, C8-10 APG with high degree of glucose oligomerization from Dow.

Preferably, the composition of the present disclosure comprises from about 0.2 to about 5%, preferably from about 0.5 to about 2% by weight of the composition of APG, preferably from about 0.5 to about 2% by weight of the composition of alkyl polyglucoside. A C8-10 alkyl polyglucoside is specially preferred for use herein.

### Emulsifier

Emulsifiers for use herein include non-ionic surfactants, in particular alkoxylated nonionic surfactants. Alkoxylated nonionic surfactants include primary C₆-C₁₆ alcohol polyglycol ether i.e. ethoxylated alcohols having 6 to 16 carbon atoms in the alkyl moiety and 4 to 30 ethylene oxide (EO) units. When referred to for example C₉₋₁₄ it is meant average carbons and alternative reference to for example EO8 is meant average ethylene oxide units.

Suitable alkoxylated nonionic surfactants are according to the formula RO-(A)ₙH, wherein: R is a C₆ to C₁₈, preferably a C₈ to C₁₆, more preferably a C₈ to C₁₂ alkyl chain, or a C₆ to C₂₈ alkyl benzene chain; A is an ethoxy or propoxy or butoxy unit, and wherein n is from 1 to 30, preferably from 1 to 20 and, more preferably from 5 to 16 even more preferably from 7 to 12. Preferred R chains of use herein are the Ca to C₂₂ alkyl chains. Even more preferred R chains of use herein are the C₁₂ to C₁₄ alkyl chains. R can be linear or branched alkyl chain.

Suitable ethoxylated nonionic surfactants of use herein are Dobanol® 91-2.5 (HLB=8.1; R is a mixture of C₉ and C₁₁ alkyl chains, n is 2.5), Dobanol® 91-10 (HLB=14.2; R is a mixture of C₉ to C₁₁ alkyl chains, n is 10), Dobanol® 91-12 (HLB=14.5; R is a mixture of C₉ to C₁₁ alkyl chains, n is 12), Greenbentine DE80 (HLB=13.8, 98 wt % C10 linear alkyl chain, n is 8), Marlipal 10-8 (HLB=13.8, R is a C10 linear alkyl chain, n is 8), Lialet® 11-5 (R is a C₁₁ alkyl chain, n is 5), Isalchem® 11-5 (R is a mixture of linear and branched C11 alkyl chain, n is 5), Lialet® 11-21 (R is a mixture of linear and branched C₁₁ alkyl chain, n is 21), Isalchem® 11-21 (R is a C₁₁ branched alkyl chain, n is 21), Empilan® KBE21 (R is a mixture of C12 and C₁₄ alkyl chains, n is 21) or mixtures thereof. Preferred herein are Dobanol® 91-5, Neodol® 11-5, Lialet® 11-21 Lialet® 11-5 Isalchem® 11-5 Isalchem® 11-21 Dobanol® 91-8, or Dobanol® 91-10, or Dobanol® 91-12, or mixtures thereof. These Dobanol®/Neodol® surfactants are commercially available from SHELL. These Lutensol® surfactants are commercially available from BASF and these Tergitol® surfactants are commercially available from Dow Chemicals.

Suitable chemical processes for preparing the alkoxylated nonionic surfactants of use herein include condensation of corresponding alcohols with alkylene oxide, in the desired proportions. Such processes are well known to the person skilled in the art and have been extensively described in the art, including the OXO process and various derivatives thereof. Suitable alkoxylated fatty alcohol nonionic surfactants, produced using the OXO process, have been marketed under the tradename NEODOL® by the Shell Chemical Company. Alternatively, suitable alkoxylated nonionic surfactants can be prepared by other processes such as the Ziegler process, in addition to derivatives of the OXO or Ziegler processes.

Preferably, said alkoxylated nonionic surfactant is a C₉₋₁₁ EO5 alkylethoxylate, C₁₂₋₁₄ EO5 alkylethoxylate, a C₁₁ EO5 alkylethoxylate, C₁₂₋₁₄ EO21 alkylethoxylate, or a C₉₋₁₁ EO8 alkylethoxylate or a mixture thereof. Most preferably, said alkoxylated nonionic surfactant is a C₁₁ EO5 alkylethoxylate or a C₉₋₁₁ EO8 alkylethoxylate or a mixture thereof.

The composition can comprise from 0.05% to 2%, preferably from 0.08% to 0.5% by weight of the composition of alkoxylated nonionic surfactant, preferably from 0.08% to 0.5% by weight of the composition of ethoxylated alcohol.

### Additional Surfactant:

The hard surface cleaning composition may comprise up to 1% by weight of an additional surfactant, preferably selected from: anionic, amphoteric, zwitterionic, and mixtures thereof. The hard surface cleaning composition can comprise from 0.01% to 1% by weight of the additional surfactant. Preferably, the composition of the present disclosure is substantially free of surfactants other than alkyl polyglycosides and alkoxylated non-ionic surfactants.

### Perfume

The composition comprises a perfume. The perfume is a mixture of odorant raw materials, such as aromatic natural oils and aromatic chemicals, which taken together form a complex scent that delivers a number of benefits. These benefits may include the coverage of product base odor, scenting the product itself, and lingering scent radiating from the surface into the air after cleaning. When the composition is sprayed, the benefit may also include the delivery of scent to the air when spraying the composition on a surface, and the delivery of scent to the air while wiping the composition on the surface. The perfume may comprise at least 3, at least 5, at least 7, at least 11, or at least 15 perfume raw materials.

As used herein, the term “perfume raw material” (or “PRM”) refers to compounds having a molecular weight of at least about 100 g/mol and which are useful in imparting an odor, fragrance, essence, or scent, either alone or with other perfume raw materials. Typical PRMs comprise inter alia alcohols, ketones, aldehydes, esters, ethers, nitrites and alkenes, such as terpene. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994). At least some of the PRMs of the present perfume include esters, aldehydes and ketones, but such PRMs are typically used in combination with other PRMs, which may include other functional groups, such as alcohols.

The PRMs may be characterized by their boiling points (B.P.) measured at the normal pressure (760 mm Hg), and their octanol/water partitioning coefficient (P), which may be described in terms of log P, determined according to the test method described in Test methods section. Based on these characteristics, the PRMs may be categorized as Quadrant I, Quadrant II, Quadrant III, or Quadrant IV perfumes, as described in more detail below. A perfume having a variety of PRMs from different quadrants may be desirable, for example, to provide fragrance benefits at different touchpoints during normal usage of the composition.

Perfume raw materials having a boiling point B.P. lower than about 250° C. and a log P lower than about 3 are known as Quadrant I perfume raw materials. Quadrant 1 perfume raw materials are preferably limited to less than 30% of the perfume composition. Perfume raw materials having a B.P. of greater than about 250° C. and a log P of greater than about 3 are known as Quadrant IV perfume raw materials, perfume raw materials having a B.P. of greater than about 250° C. and a log P lower than about 3 are known as Quadrant II perfume raw materials, perfume raw materials having a B.P. lower than about 250° C. and a log P greater than about 3 are known as a Quadrant III perfume raw materials. Suitable Quadrant I, II, III, and IV perfume raw materials are disclosed in U.S. Pat. No. 6,869,923 B1.

The perfume of the composition of the present disclosure raw materials of the perfume comprises at least 10% by weight of the perfume of perfume raw materials comprising an ester functionality, an aldehyde functionality, a ketone functionality or a mixture thereof. Preferably, the perfume comprises at least 10% by weight of the perfume of perfume raw materials comprising an ester functionality. Preferably, the perfume comprises at least 5% by weight of the perfume of perfume raw materials comprising an aldehyde functionality and preferably at least 5% by weight of the perfume of perfume raw materials comprising a ketone functionality. More preferably, the perfume comprises at least 25% by weight of the perfume of a mixture of perfume raw materials comprising ester, aldehyde and ketone functionalities.

### Perfume Raw Materials Comprising an Ester Functionality

Examples of perfume raw materials comprising an ester functionality include acetates, anthranilates, benzoates, butyrates, cinnamates, formates, isobutyrates, phenylacetates and propionates. Some ester-containing perfume raw materials include: anisyl acetate, benzyl benzoate, cedryl formate, cinnamyl isobutyrate, citronellyl acetate. citronellyl formate, dihydrocarvyl acetate, diheptyl acetate, ethyl phenyl acetate, furfuryl phenyl acetate, geranyl benzoate, geranyl butyrate, hexyl caproate, isobutyl phenol acetate, isononyl acetate, isononyl propionate, linalyl acetate, linalyl formate, phenoxy ethyl isobutyrate, benzyl acetate, tricyclodecanyl acetate, tricyclodecanyl propionate, geranyl acetate, phenyl ethyl acetate, pseudo linalyl acetate and methyl benzoate and mixtures thereof.

### Perfume Raw Materials Comprising an Aldehyde Functionality

Perfume raw materials comprising an aldehyde functionality may comprise from about eight to about twelve carbon atoms; such PRMs may be called C8-C12 aldehydes.

As examples, the structures of two aldehydes, each having twelve carbon atoms (e.g., both are C12 aldehydes), are provided below: dodecanal (a linear aldehyde-containing PRM; also known as dodecyl aldehyde or lauryl aldehyde) and 2-methylundecanal (a non-linear aldehyde-containing PRM; also known as methyl nonyl acetaldehyde, or “MNA”) are shown below.

| PRM Name | Structure |
|---|---|
| dodecanal (linear) | 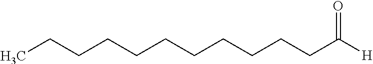 |
| 2-methylundecanal (or “MNA”) (non-linear) | 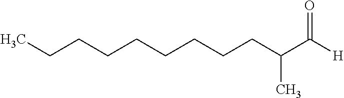 |

The perfume may comprise an aldehyde-containing perfume raw material (or residue thereof) selected from the group consisting of: decyl aldehyde, intreleven aldehyde, lauric aldehyde, methyl nonyl acetaldehyde, nonyl aldehyde, octyl aldehyde, undecyl aldehyde, undecylenic aldehyde, trans-2 hexenal, aqual, pinyl isobutyraldehyde alpha, calypsone, nironal, noreenal, nympeal, cyclamen aldehyde, ligustral, neo hivernal, p.t. bucinal (lilial), trans-2-docecenal, tangerinal, anisic aldehyde, iso hexenyl cyclohexenyl carboxaldehyde, melonal, citral, dupical, vanillin, ethyl vanillin, helional, heliotropin, hexyl cinnamic aldehyde, hydroxycitronellal, citronellal, floralozone, starfleur, 4,7-Methano-1H-indene-2-carboxaldehyde, octahydro-5-methoxy-(Scentenal® 981810), adoxal, mefranal, florhydral, bergamal, canthoxal, cinnamic aldehyde, citrathal, citronellyl oxyacetaldehyde, cyclemax, floral super, 3-Cyclohexene-1-propanal,beta,4-dimethyl-(Liminal® 955374), lyral, phenyl acetaldehyde, pino acetaldehyde, trans-4-decenal, vernaldehyde, Octahydro-1H-4,7-methanoindene-5-carbaldehyde (Vertral®), and mixtures thereof.

### Perfume Raw Materials Comprising a Ketone Functionality

Perfume raw materials comprising a ketone functionality include, for example, methyl beta-naphthyl ketone, musk indanone (1,2,3,5,6,7-Hexahydro-1,1,2,3,3-pentamethyl-4H-inden-4-one), tonalide (6-Acetyl-1,1,2,4,4,7-hexanethyltetralin), alpha-damascone, beta-damascone, delta-danascone, iso-damascone, damascenone, methyl dihydrojasmonate, menthone, carvone, camphor, kovone (3,4,5,6,6-pentamethylhept-3-en-2-one). Fenchone, alpha-ionone, beta-ionone, gamma-methyl ionone, fleuramone (2-heptylcyclopene-tanone), dihydrojasmone, cis-jasmone, iso-E-Super (1-(1,2,3,4,5,6 J,8-octahydro-2,3,8,8-tetramethyl-2-naphthalenyl)-ethan-1-one (and isoniers), methyl cedrenyl ketone, acetophenone, methyl acetophenone, para-methoxy acetophenone, methyl beta-naphthyl ketone, benzyl acetone, benzophenone, para-hydroxyphenyl butanone, Selenium ketone (3-methyl-5-propyl-2-cyclohexenone), 6-isopropyldecahydro-2-naphthone, dimethyloctenone, Fresnel menthe (2-butan-2-yl-cyclohexan-1-one), 4-(1-ethoxyvinyl)-3,3,5,5-tetramethylcyclohexanone, methylheptenone, 2-(2-(4-methyl-3-cyclohexen-1-yl) propyl) cyclopentanone, 1-(p-Menthen-6 (2) yl)-1-propanone, 4-(4-hydroxy-3-methoxyphenyl)-2-butanone, 2-Acetyl-3,3-dimethylnorbornane, 6,7-Dihydro-1,1,2,3,3-pentamethyl-4 (5H)-indanone, 4-damascol, Dulcinyl (4-(1,3-benzodioxol-5-yl) butan-2-one), hexalone (1-(2,6,6-trimethyl-2-cyclohexen-1-yl)-1,6-heptadien-3-one), IsocyclemonE (2-acetonaphthone-1,2,3,4,5,6,7,8-octahydro-2,3,8,8-tetramethyl), methyl nonyl ketone, methyl cyclocitrone, methyl lavendel ketone, orivone (4-tert-amylcyclohexanone), 4-tert-butylcyclohexanone, Delphon (2-pentylcyclopentanone), muscone (CAS 541-91-3), neobutenone (1-(5,5-dimethyl-1-cyclohexenyl)pent-4-en-1-one), plicatone (CAS 41724-19-0), veloutone (2,2,5-trimethyl-5-pentylcyclopentan-1-one), 2,4,4,7-tetramethyloct-6-en-3-one and tetrameran (6,10-dimethylundecen-2-one).

Perfume raw materials comprising a ketone functionality include alpha-damascone, beta-damascone, gamma-damascone, deltadamascone, alpha-ionone, beta-ionone, gamma-ionone, delta-ionone, beta-damascenone, 3-methyl-5-propyl-2-cyclohexen-1-one, 1 (6),8-P-menthadien-2-one, 2,5-dimethyl-5-phenyl-1-hexen-3-one, 1-(5,5-dimethyl-1-cyclohexen-1-yl)-4-penten-1-one, 8 or 10-methyl-alpha-ionone, 2-octenal, 1-(2,2,3,6-tetramethyl-1-cyclohexyl)-2-buten-1-one, 4-(2,2,3,6-tetramethyl-1-cyclohexyl)-3-buten-2-one, 2-cyclopentadecen-1-one, nootkatone, cinnamic aldehyde, 2,6,6-trimethyl-bicyclo[3.1.1]heptane-3-spiro-2′cyclohexen-4′-one, ethyl 2,4-deca-dienoate, ethyl 2-octenoate, methyl 2-nonenoate, ethyl 2,4-undecadienoate and methyl 5,9-dimethyl-2,4,8-decatrienoate.

The composition may comprise from 0.05% to 1%, or from 0.1% to 0.8% by weight of perfume.

### Optional Ingredients:

The liquid hard surface cleaning compositions may comprise a variety of optional ingredients depending on the technical benefit aimed for and the surface treated. Suitable optional ingredients of use herein include builders, polymers, buffers, bactericides, hydrotropes, colorants, stabilisers, radical scavengers, abrasives, soil suspenders, brighteners, anti-dusting agents, dispersants, dye transfer inhibitors, pigments, silicones and/or dyes.

Preferred compositions comprise:
a) from about 0.2% to about 2% by weight of the composition of alkyl polyglycoside surfactant, preferably an alkyl polyglucoside, more preferably a C8-10 alkyl polyglucoside;
b) from about 0.05% to about 0.3% by weight of the composition of ethoxylated alcohol, preferable C12-C14 ethoxylated alcohol with a degree of ethoxylation of from 5 to 15, preferably from 8 to 12;
c) from 0.1 to 2% by weight of the composition of perfume.

The composition can be in the form of a ready-to-use composition or in the form of a concentrated composition.

A prefer composition for use herein, especially as a sprayable composition, comprises:
a) from about 0.5 to 2% by weight of the composition of the alkyl polyglycoside, preferably an alkyl polyglucoside, more preferably a C8-10 alkyl polyglucoside;
b) from about 0.1 to 0.5% by weight of the composition of an emulsifier, preferably a non-ionic surfactant, more preferably an alcohol ethoxylated; and
c) from about 0.1 to 0.5% by weight of the composition of the perfume.

A prefer composition for use herein, especially as a concentrated composition to be diluted in water before use, comprises:
a) from about 5 to 20% by weight of the composition of the alkyl polyglycoside, preferably an alkyl polyglucoside, more preferably a C8-10 alkyl polyglucoside;
b) from about 1 to 5% by weight of the composition of an emulsifier, preferably a non-ionic surfactant, more preferably an alcohol ethoxylated; and
c) from about 1 to 5% by weight of the composition of the perfume.

### Method of Cleaning a Surface:

The preferred method of cleaning comprises the steps of:
a) contacting the surface with the composition of the present disclosure;
b) wiping the surface.
   Preferably, the method of the present disclosure does not require rinsing.

The composition can be a “ready-to-use” composition, where dilution is not necessary. Such ready-to-use compositions can be comprised in a spray container.

In another preferred embodiment of the present disclosure said method of cleaning a hard surface includes the steps of applying, preferably spraying, said liquid composition onto said hard surface, leaving said liquid composition to act onto said surface for a period of time to allow said composition to act, with or without applying mechanical action.

### Methods:

### pH Measurement:

The pH is measured on the neat composition, at 25° C., using a Sartarius PT-10P pH meter with gel-filled probe (such as the Toledo probe, part number 52 000 100), calibrated according to the instructions manual.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

### EXAMPLE

Three compositions comprising C8-C10 alkylpolyglucoside (APG) were made by simple mixing (Compositions A* to C). Composition A* was made using a C8-C10 alkylpolyglucoside solution having pH 12. Composition B* was made using a C8-C10 alkylpolyglucoside solution having pH 7. Composition C was made by neutralizing a C8-C10 alkylpolyglucoside solution having an initial pH 12 with citric acid to bring the pH down to pH 7.

| | A* | B* | C |
|---|---|---|---|
| | wt % | wt % | wt % |
| Monoethanolamine | 0.3 | 0.3 | 0.3 |
| Sodium carbonate | 0.1 | 0.1 | 0.1 |
| Propylene glycol n-butyl ether | 0.2 | 0.2 | 0.2 |
| C8-C10 APG (pH 12) | 0.12 | 0.12 | 0.12 |
| C8-C10 APG (pH 7) | 0.15 | 0.15 | 0.15 |
| C8-C10 APG (pre-neutralized | — | — | 1.0 |
| with citric acid to pH 7) | | | |
| C12-14 EO 11¹ | 0.2 | 0.2 | 0.2 |
| Perfume (as show in table | 0.2 | 0.2 | 0.2 |
| below) | | | |
| Final composition pH | 11 | 11 | 11 |

*Comparative
¹nonionic surfactant commercially available from Sasol

The composition of the perfume is shown in the table below.

| | wt % |
|---|---|
| Terpenes | 34.4 |
| Ester | 19.9 |
| Aldehydes | 7.2 |
| Ether/ketone | 14.9 |
| Alcohol | balance |

The compositions were stored for 2 weeks at 25° C. in PET bottles. The perfume was extracted via automatic hexane extraction. The components of the perfume were analysed via gas chromatography mass spectrometry using an Agilent 7890B GC+5977B MSD. The table below shows the loss of perfume of compositions A* to C after two weeks.

| Loss of perfume (wt %) | A* | B* | C |
|---|---|---|---|
| Terpenes | 52.2 | 34 | 34 |
| Ester | 84.7 | 65.3 | 24.7 |
| Aldehydes | 74.9 | 56.8 | 59.8 |
| Ether/ketone | 63.8 | 16 | 7 |
| Alcohol | balance | balance | balance |

The compositions made with APG at neutral pH shows better stability of esters, ether and ketones versus a composition made from an alkaline APG. A composition made by pre-neutralization of alkaline APG with citric acid shows superior stability of esters, ethers and ketone versus both a composition made with APG neutral pH per design and composition made from alkaline APG.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any present disclosure disclosed or claimed herein or that it alone, or in any combination with any other reference or references, teaches, suggests or discloses any such present disclosure. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

What is claimed is:

1. A process for making a composition, comprising:
providing a concentrated aqueous solution comprising from about 30 to about 80 wt. % alkyl polyglycoside;
adding a neutralizing agent to the concentrated aqueous solution to form a mixture; and
adding perfume to the mixture to form the composition, wherein:
the composition comprises:
alkyl polyglycoside surfactant; and
at least about 0.1% perfume by weight of the composition,
the perfume comprises at least about 10 wt. % perfume raw material,
the perfume raw material comprises an ester functionality, an aldehyde functionality, a ketone functionality, or combinations thereof, and
the composition has a pH equal to or greater than about 7, as measured at about 25° C.

2. The process according to claim 1, wherein the concentrated aqueous solution has a pH greater than about 11 as measured at 25° C.

3. The process according to claim 1, wherein the addition of the neutralizing agent to the concentrated aqueous solution reduces the pH of the solution by at least about 3 pH units.

4. The process according to claim 1, wherein the addition of the neutralizing agent to the concentrated aqueous solution reduces the pH of the solution by at least about 4 pH units.

5. The process according to claim 1, wherein the pH of the mixture is from about 6.5 to about 8.5.

6. The process according to claim 1, wherein the neutralizing agent comprises citric acid.

7. The process according to claim 1, wherein the perfume comprises at least about 10% by weight thereof of perfume raw materials comprising an ester functionality.

8. The process according to claim 1, wherein the perfume comprises at least about 5% by weight thereof of perfume raw materials comprising an aldehyde functionality and preferably at least about 5% of perfume raw materials comprising a ketone functionality.

9. The process according to claim 1, wherein the perfume comprises at least about 5% by weight thereof of perfume raw materials comprising an aldehyde functionality and at least about 5% of perfume raw materials comprising a ketone functionality.

10. The process according to claim 1, wherein the perfume comprises at least about 25% by weight thereof of a mixture of perfume raw materials comprising ester, aldehyde and ketone functionalities.

11. The process according to claim 1, wherein the perfume comprises a non-ionic surfactant.

12. The process according to claim 1, wherein the composition has a pH equal to or greater than about 10 as measured at about 25° C.

13. The process according to claim 1, further comprising:
making an alkaline aqueous solution,
adding the composition to the alkaline aqueous solution to form an aqueous composition, and
adding dyes to the aqueous composition.

14. A composition comprising:
alkyl polyglycoside surfactant; and
at least about 0.1% perfume by weight of the composition, wherein:
the perfume comprises at least about 10 wt. % perfume raw material,
the perfume raw material comprises an ester functionality, an aldehyde functionality, a ketone functionality, or combinations thereof, and
the composition has a pH equal to or greater than about 7, as measured at about 25° C.

15. The composition according to claim 14, further comprising an emulsifier wherein the alkyl polyglycoside surfactant and the emulsifier are present in a weight ratio of at least 4:1.

16. The composition according to claim 14, comprising an emulsifier wherein the emulsifier comprises a non-ionic surfactant.

17. The composition according to claim 14, comprising an alkoxylated alcohol.

18. The composition according to claim 14, comprising an alkoxylated alcohol comprising from about 6 to about 16 carbon atoms and from about 2 to about 12 alkoxy groups.

19. The composition according to claim 14, wherein the composition comprises:
from about 0.5 to about 2% by weight of the composition of the alkyl polyglycoside;
from about 0.1 to about 0.5% by weight of the composition of an emulsifier, preferably a non-ionic surfactant, more preferably an alcohol ethoxylated; and
from about 0.1 to about 0.5% by weight of the composition of the perfume.

20. The composition according to claim 14, wherein the composition comprises:
from about 5 to about 20% by weight of the composition of the alkyl polyglycoside;
from about 1 to about 5% by weight of the composition of an emulsifier, preferably a non-ionic surfactant, more preferably an alcohol ethoxylated; and
from about 1 to about 5% by weight of the composition of the perfume.
